SEQUENCE LISTING (14 sequences)

SEQ ID NO: 1 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 2 - protein, 196 aa, Homo sapiens
MADGQMPFSCHYPSRLRRDPFRDSPLSSRLLDDGFGMDPFPDDLTASWPDWALPRLSSAWPGTLRSGMVPRGPTATARFGVPAEGRTPPPFPGEPWKVCVNVHSFKPEELMVKTKDGYVEVSGKHEEKQQEGGIVSKNFTKKIQLPAEVDPVTVFASLSPEGLLIIEAPQVPPYSTFGESSFNNELPQDSQEVTCT

SEQ ID NO: 3 - protein, 182 aa, Homo sapiens
MSGRSVPHAHPATAEYEFANPSRLGEQRFGEGLLPEEILTPTLYHGYYVRPRAAPAGEGSRAGASELRLSEGKFQAFLDVSHFTPDEVTVRTVDNLLEVSARHPQRLDRHGFVSREFCRTYVLPADVDPWRVRAALSHDGILNLEAPRGGRHLDTEVNEVYISLLPAPPDPEEEEEAAIVEP

SEQ ID NO: 4 - protein, 173 aa, Homo sapiens
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 5 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 6 - protein, 160 aa, Homo sapiens
MEIPVPVQPSWLRRASAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAK

SEQ ID NO: 7 - protein, 154 aa, Homo sapiens
MALSLFGGRRSNVFDPFSLDIWDPFEGFSAVANVPPSARETTAFATARIDWKETPEAHIFKADLPGLKKEEVKVEVEDGNVLQISGERSKEHEEKNDKWHRVERSCGKFLRRFRLPENAKVEQVKANMENGVLTVIVPKEEQKKTEVKSIEISG

SEQ ID NO: 8 - protein, 150 aa, Homo sapiens
MAKIILRHLIEIPVRYQEEFEARGLEDCRLDHALYALPGPTIVDLRKTRAAQSPPVDSAAETPPREGKSHFQILLDVVQFLPEDIIIQTFEGWLLIKAQHGTRMDEHGFISRSFTRQYKLPDGVEIKDLSAVLCHDGILVVEVKDPVGTK

SEQ ID NO: 9 - protein, 170 aa, Homo sapiens
MSHRTSSTFRAERSFHSSSSSSSSSTSSSASRALPAQDPPMEKALSMFSDDFGSFMRPHSEPLAFPARPGGAGNIKTLGDAYEFAVDVRDFSPEDIIVTTSNNHIEVRAEKLAADGTVMNTFAHKCQLPEDVDPTSVTSALREDGSLTIRARRHPHTEHVQQTFRTEIKI

SEQ ID NO: 10 - protein, 159 aa, Homo sapiens
MQRVGNTFSNESRVASRCPSVGLAERNRVATMPVRLLRDSPAAQEDNDHARDGFQMKLDAHGFAPEELVVQVDGQWLMVTGQQQLDVRDPERVSYRMSQKVHRKMLPSNLSPTAMTCCLTPSGQLWVRGQCVALALPEAQTGPSPRLGSLGSKASNLTR

SEQ ID NO: 11 - protein, 151 aa, Homo sapiens
MSIVRRTNVFDPFADLWADPFDTFRSIVPAISGGGSETAAFANARMDWKETPEAHVFKADLPGVKKEEVKVEVEDGNVLVVSGERTKEKEDKNDKWHRVERSSGKFVRRFRLLEDAKVEEVKAGLENGVLTVTVPKAEVKKPEVKAIQISG

SEQ ID NO: 12 - DNA, 537 bp, Homo sapiens
tagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgggaagagaagcctgctgtcaccgcagcccccaagaaataga

SEQ ID NO: 13 - DNA, 537 bp, Homo sapiens
tagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaataga

SEQ ID NO: 14 - protein, 12 aa, Homo sapiens
EEKPAVTAAPKK

(19) United States
(12) Patent Application Publication
(10) Pub. No.: US 2005/0148058 A1
(43) Pub. Date: Jul. 7, 2005

(54) Protein stabilization

(76) Inventor: Roy Quinlan, Dundee (GB)

Correspondence Address: WINSTON & STRAWN LLP, 1700 K STREET, N.W., WASHINGTON, DC, 20006, US

(21) Appl. No.: 10/969,925

(22) Filed: Oct. 22, 2004

Related U.S. Application Data
(63) Continuation of application No. PCT/GB03/01721, filed on Apr. 23, 2003.

(30) Foreign Application Priority Data
Apr. 23, 2002 (GB) 0209334.2

(51) Int. Cl. C12P 21/06; C12N 9/64; C12N 9/06; C07K 14/62

(52) U.S. Cl. 435/191; 530/303; 435/226

(57) ABSTRACT

The present invention relates to protein chaperones, such as hybrid chaperones and methods for stabilizing proteins and protein activities comprising adding said protein chaperone to the protein. The present invention also provides a stabilized protein formulation comprising said protein chaperone associated with a protein and further relates to the enhancement of native chaperone activity by making hybrid protein chaperones.

### CROSS-REFERENCE TO RELATED APPLICATIONS

This application is a continuation of International Application No. PCT/GB03/01721, filed Apr. 23, 2003, the entire content of which is expressly incorporated herein by reference thereto.

### FIELD OF THE INVENTION

The present invention relates to protein chaperones, such as hybrid chaperones and methods for stabilizing proteins and protein activities comprising adding said protein chaperone to the protein. The present invention also provides a stabilized protein formulation comprising said protein chaperone associated with a protein. The present invention further relates to the enhancement of native chaperone activity by making hybrid protein chaperones.

### BACKGROUND OF THE INVENTION

Protein chaperones can be subdivided into 4 major protein families on the basis of their primary sequence and chaperone properties. These include HSP90, HSP70, HSP60 and sHSP protein classes. The HSP prefix stands for “Heat Shock Protein” and indicates how these proteins were first discovered—they are very prominent in stressed cells. Here they facilitate the folding of a whole range of different protein targets and help maintain a range of different protein activities in cells (Saibil, 2000).

Over the last decade it has emerged that the protection afforded by sHSPs is due to their ability to bind proteins that are in the process of aggregating (Lindner et al., 2001). Such destabilized proteins put the whole cell at risk because they provide a focus for the precipitation of cellular proteins, whether they themselves are in the process of unfolding or not (Schubert et al., 2000). So sHSPs are a key part of the cellular defense mechanism to prevent such a catastrophe, protecting the cell from this inner danger.

Subsequent research has shown that protein chaperones including the sHSPs are present and active in normal unstressed cells, so their activity is not just important in stressed cells (Saibil, 2000). The high intracellular protein concentrations in cells (Ellis and Hartl, 1999) and the high failure rate of the protein translation machinery (Schubert et al., 2000) are conditions that favor protein misfolding and consequently protein aggregation. Chaperone activity is therefore an essential part of cell viability.

EP0599344A1 discloses the efficacy of HSP2S, a mammalian sHSP, to stabilize proteins in vitro. In particular, this document addresses protein aggregation, with only one example of stabilizing an enzyme activity.

However, since the filing date of EP0599344A1, there have been three fundamental advances, which cast doubt on the teaching of EP0599344A1.

The first important advance is the identification of eleven different sHSPs in mammalian cells alone (Table 1).

TABLE 1

| SHSP | Tissue Location | Identified Physiological Target |
|---|---|---|
| αA-crystallin | Lens | Intermediate Filaments |
| αB-crystallin | Widespread expression | Intermediate Filaments/Actin |
| HSP27 | Widespread expression | Intermediate Filaments/Actin |
| HSP20 | Muscle & endothelial | Unknown |
| | cells | |
| MKBP | Muscle | MDPX |
| HSPS3 | Muscle | Unknown |
| HSPS4 | Muscle | Unknown |
| HSPS5 | Muscle | Unknown |
| evHSP | Muscle | Filamin |
| HSPB8 | Muscle | |
| HSPB9 | Testis | |

As three members have been discovered in the last two years (Kappe et al., 2001; Krief et al., 1999}, it is unwise to conclude that this list is now complete. Within the wider small heat shock protein family new members are being discovered with limited sequence homology, but conserved functional and structural homology. Only some have been linked to specific cellular functions and this list is growing all the time. For instance, some are concerned with maximizing enzymatic (kinase) activity (MKBP) (Suzuki et al., 1998), while others (HSP27 and αB-crystallin) stabilizes multi-protein complexes in cells (Perng et al., 1999a). The protein αB-crystallin has also been shown to bind to DNA (Pietrowski et al., 1994) and to be involved in genome stability (Andley et al., 2001), while both HSP27 (Mehlen et al., 1996) and αB-crystallin (Kamradt et al., 2001) have been shown to help protect cells against apoptosis-inducing agents. HSP27 and α-crystalline can inhibit amyloid fibril polymerization (Hatters et al., 2001; Kudva et al., 1997). HSP27 and αB-crystallin can also increase the activity of the MKBP-target kinase in vitro, but they are not as good as MKBP itself (Suzuki et al., 1998). In fact, one sHSP, HSPB8 is a ser/thr kinase that is essential for keratinocyte cell growth (Aurelian et al., 2001). On the other hand, HSP27 cannot inhibit platelet aggregation, a function of HSP20 (Niwa et al., 2000}.

Thus the second important advance is that the various sHSPs are not equivalent in their physiological roles. The sHSPs are involved in a very broad range of activities from stabilization of the cytoskeleton to genome stability. These observations show that in vivo the different sHSPs have specialist tasks. The in vitro chaperone assays also show that different sHSPs function with variable efficacy using non-physiological substrates (van de Klundert et al., 1998). The wide range ot substrates used in such in vitro assays do suggest, however, that sHSPs have potential application in stabilizing a wide range of proteins, but optimization for commercial viability is required in each case.

The third important advance to appear in the literature (Reddy et al., 2000) is that the activity profiles of the different sHSPs can vary with temperature, another possibility not considered in EP0599344A1. Thus, from these new developments it is impossible to pick a single sHSP as the “absolute best” representative of the whole class for all possible functions, as was suggested in EP0599344A1.

It is an object of the present invention to obviate and/or mitigate at least one of the aforementioned disadvantages.

### SUMMARY OF THE INVENTION

The present invention is based in part on the inventor's observations that creating hybrid sHSPs by replacing one or more regions of a sHSP with a similar region(s) from another sHSP can improve the activity as compared to native sHSPs, which, it could have been argued that changing a sequence previously selected under evolutionary pressure would reduce activity.

Thus, in a first aspect of the present invention there is provided a hybrid protein chaperone for stabilizing proteins and/or protein activities.

“Hybrid”, according to the present invention, is understood to mean any macromolecule composed of two or more portions of different origins. Typically, said hybrid comprises a macromolecule with two or more portions, wherein at least one portion has been replaced with a similar portion from a different origin. For example, said macromolecule may be DNA, which it will be understood may be translated into a hybrid protein product, preferably a hybrid protein chaperone. It will be understood that said replacement of the portion of macromolecule will be done such that it will remain ‘in-frame’ with the rest of the macromolecule allowing translation of a full-length protein product, for example.

Typically, said portions may include functional homologues thereof. Thus, without wishing to be bound by theory, functional homologues according to the present invention should be understood to mean regions of protein or nucleic acid sequence conserved throughout a family of protein chaperones, comprising, for example, structural domains which retain the function of chaperone properties. In more detail, structural domains may be determined based on structure-function studies published for the mammalian sHSP family (Kim et al., 1998, van Montfort et al., 2001). From such publications and the sequence alignments for the whole sHSP family available in the public domain (de Jeng et al., 1998; Kappe et al., 2002; see sequence alignment profile from 140 sHSPs available at ftp.cmbi.kun.nl/pub/molbiol/kappe/), regions of homology have been correlated with specific structural domains in the protein. All HSPs mentioned in the above references are encompassed within the present invention as being suitable for generating hybrid chaperones. So sHSPs in general fit a model of a central domain, called the α-crystallin domain, flanked by N- and C-terminal regions that are variable in sequence, length and structure (de Jong et al., 1998). It should be noted, however, that some chaperones, including small heat shock proteins, are related to the sHSP family only by functional and structural similarities rather than sequence (de Jong et al 1998; van Montfort et al., 2001). The HSP90 cochaperone p23 although sequentially unrelated is topologically similar to HSP16.9 and offers a strategy to further modify the sHSPs to generate functional monomeric proteins (van Montfort et al, 2001). Therefore, it should also be understood that functional homologue may mean chaperone proteins/nucleic acid sequences or portions thereof which do not retain high sequence similarity to protein chaperone family but do retain chaperone family functional properties.

Preferably said portions according to the present invention are generally selected from the central domain (α-crystallin domain), N-terminal region or C-terminal region of the sHSP family proteins.

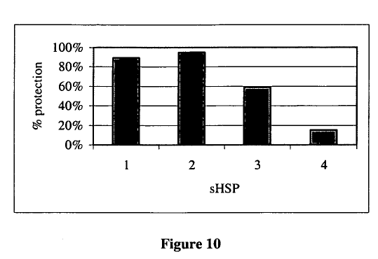

It should be understood that the N-terminal region according to the present invention comprises up to 5, 10, 15, 20, 25, 30, 35, 40, 45 or 50 amino acid residues from the start codon, methionine of anyone of the protein chaperones of the present invention and/or as shown in FIG. 10.

It should be understood that the C-terminal region according to the present invention comprises up to 5, 10, 15, 20, 25, 30, 35, 40, 45 or 50 amino acid residues prior to the stop codon of anyone of the protein chaperones of the present invention and/or as shown in FIG. 10. For example, the 12 amino acid portion located between conserved amino acid site E164 (Accession No. P02511) and the stop codon of αB-Crystallin as shown in FIG. 10.

Empirical studies coupled with genetic studies have nevertheless revealed sub-domains and specific residues that are critical to function for some sHSPs. Thus, optionally said portions may comprise said sub-domains or residues.

In more detail, work on αB-crystallin has revealed the importance of Arginine 120, a residue that is highly conserved throughout the whole sHSP family (Perng et al., 1999b). Mutation of this residue to a glycine causes the inherited diseases of cardiomyopathy and cataract (Vicart et al., 1998). Mutation of the equivalent residue to cysteine in αA-crystallin, a related sHSP, also causes cataract (Litt et al., 1998). However, current structure-function predictions still require empirical verification (van Montfort et al., 2000). For example, in other studies published recently, the importance of the C-terminal region to αB-crystallin is apparent (Berry et al., 2001). A point mutation that altered the coding frame and changed the sequence of the C-terminal region has been shown to be the genetic basis of another inherited cataract. These data provide the first clear evidence that this region is important in αB-crystallin. Structural studies with HSP25 have also shown that this region can influence chaperone activity (Lindner et al., 2000). In some mammalian sHSPs, however, this region is virtually non-existent (Kappe at al.,: 2001).

A hybrid protein chaperone according to the present invention is understood to mean a protein chaperone wherein a portion of said chaperone is replaced with a similar portion from a chaperone of a different origin. For example, the C-terminal region of one chaperone may be replaced with a corresponding C-terminal region of any of a number of other protein chaperones. Preferably, the protein chaperone activity of said hybrid is optimized for its application. Without wishing to be bound by theory it is understood that different hybrids may have different efficacies for each application.

In one embodiment of the present invention, said hybrid is prepared by swapping a C-terminal portion of αB-crystallin with a C-terminal portion of BSP27 to create a hybrid, namely αB-HSP27. Thus it should be understood that αB-HSP27 comprises the N-terminus and central portion of αB-crystallin and C-terminal tail of HSP27.

Conveniently, said hybrid is constructed using known techniques. For example, a restriction enzyme site may be introduced upstream of the C-terminal region of a protein chaperone gene using site-directed mutagenesis, well-known to those skilled in the art (see for example Sambrook et al., 2001) allowing the different N- and C-terminal regions of various gene products to be produced by digestion via an appropriate restriction enzyme. Thus, a digested gene portion encoding a C-terminal region of a protein chaperone may be ligated into a similarly restriction enzyme-digested “parent” protein chaperone gene encoding an N-terminal region, such that the ligated portion is in-frame with the digested gene it has been ligated to. This hybrid gene construct may then be cloned (ligated) into an appropriate vector for protein expression of the hybrid protein chaperone gene. Thus, the hybrid protein chaperone gene product should give rise to a functional protein chaperone i.e. capable of stabilizing a protein or protein activity. This may easily be tested for using in vitro tests on the desired protein, by conducting a functional test for the protein, with and without the protein chaperone and under conditions which are shown to destabilize the protein in the absence of a protein chaperone. It will be understood to the skilled man that said hybrids may he constructed using other methods known in the art, for example, using PCR techniques or the use of restriction enzyme digest of naturally occurring restriction enzyme sites.

A protein chaperone according to the present invention including functional homologue thereof is a protein or portion thereof demonstrating chaperone-like activity, protecting heterologous proteins from insults by preventing protein aggregation, preserving activity and/or restoring activity to previously damaged proteins. Typically, the protein is a member of the chaperone protein family, also named heat shock proteins (HSPS), derived from prokaryotic or eukaryotic organisms including HSP90. HSP70, HSP60 and small HSPs. Preferably, said protein chaperone belongs to the family of small Heat Shock Proteins (sHSPs) including crystallins, for example, αA-crystallin or αB-crystallin. Members of the mammalian sHSP family include HSP27, HSP20, MKBP, HSPB3, HSPB4, HSPB5, cvHSP, HSPB8 and HSPB9. It will be understood that protein-encoding nucleic acid sequence and/or amino acid sequence of the protein chaperones of the present invention include members of the protein chaperone family that may have residues therein substituted, added or deleted while maintaining functional activity.

Typically, sHSPs form large protein complexes and function by arresting protein unfolding. In some cases the refolding of the stabilized protein substrate requires other protein chaperones, e.g. HSP70. Advantageously, mammalian sHSPs do not require an energy source (e.g., ATP or GTP) in order to function as protein stabilizers.

In a further aspect of the present invention there is provided a stabilized protein formulation comprising at least one protein associated with a hybrid protein chaperone according to the present invention. Typically, the ratio of protein to hybrid protein chaperone in said formulation is in the region of 25:1 to 1:100 such as, 1:0.0625 to 1:40.

In a yet further aspect of the present invention there is provided q method for stabilizing proteins and/or protein stabilities in an aqueous solution comprising adding a hybrid protein chaperone to said aqueous solution.

The method according to the present invention can be applied to any protein. Preferably it is applied to proteins which have a tendency to aggregate, whether due to their temperature sensitivity or other reasons. Thus, an important field of application for the method according to the present invention is in the field of bio-diagnostics, particularly to increase the product shelf-life and/or stability of protein reagents used. An example of a protein reagent is homocysteine desulphurase.

For example, said protein reagents include reagents used in immunoassay diagnostic kits such as ELISA including antibodies, antibody fragments and antibody conjugates. particularly, said protein reagents include antibody conjugates that possess a covalently linked enzyme reporter, for example, horseradish peroxidase (HRP), alkaline phosphatase (ALP), or luciferase.

Further applications of the present invention include the use of a hybrid protein chaperone according to the present invention as an agent to prevent. protein aggregation or as an inhibitor of cell death and genome stability pathways.

Conveniently, the method and the hybrid protein chaperones of the present invention may also be applied to the area of quality control (QC) Test Development and antigen stabilization. For example, in QC Tests the hybrid protein chaperones may be used to recognize proteins that are in the process of unfolding. This may include the recognition of multiprotein complexes, for example the formation of filament structures and, in particular, amyloid formation. In a similar manner, the method according to the present invention may be used to recognize and stabilize antigens that are detected using the commercially available diagnostic kits. This would be advantageous due to the fact that proteins (antigens) become hypersensitive to proteolytic attack when they become unfolded and any stabilization by said hybrid protein chaperones would increase the shelf-life of such proteins.

“Stabilization”, according to the present invention, is understood to mean the prevention or arresting of the unfolding process and preserving protein activity/function. Typically, this is achieved, for example, by assisting proteins to fold correctly and maintaining the proteins in a folded conformation, which preserves function or preventing the proteins from aggregating.

It will be understood that, for optimum efficacy, specific hybrid protein chaperones may be needed for specific tasks.

Thus, in a further embodiment of the present invention the method uses αB-crystallin hybrids in the protection of enzyme activity, such as luciferase activity. Preferably, it uses αB-HSP27 protein chaperone in hybrid stabilization, such as stabilization of insulin, HRP conjugate, luciferase, homocysteine desulphurase and antibodies.

In a yet further aspect of the present invention there is provided use of hybrid protein chaperones according to the present invention in the treatment of disease involving altered protein conformations. Such diseases include, for example, cardiomyopathies, cataract and several neurodegenerative diseases.

In a further aspect, the present invention provides the use of hybrid protein chaperones for the manufacture of a medicament for the treatment of disease involving altered protein conformation.

The present invention provides yet further aspects, which are set out below:

Use of HSP17.5, α-crystallin, HSP27, αB-crystallin, αA-crystallin or HSP25 for stabilizing insulin.

A method for stabilizing insulin in an aqueous solution comprising adding HSP17.5, α-crystallin, HSP27, αB-crystallin, αA-crystallin or HSP25 to said aqueous solution.

Preferably, HSP17.5 is used to stabilize insulin at 37° C. More preferably, HSP27 or α-crystallin is used to stabilize insulin at 44° C.

Use of αA-crystallin, αB-crystallin. α-crystallin, HSP25 or HSP27 for stabilizing citrate synthase.

A method for stabilizing citrate synthase in an aqueous solution comprising adding αA-crystallin, αB-crystallin, α-crystallin, HSP25 or HSP27 to said aqueous solution.

Preferably, αA-crystallin, αB-crystallin or α-crystallin is used to stabilize citrate synthase. More preferably, α-crystallin is used to stabilize citrate synthase at 50° C.

Use of HSP17.5, HSP27 or α-crystallin for stabilizing luciferase.

A method for stabilizing luciferase in an aqueous solution comprising adding HSP17.5, HSP27 or α-crystallin to said aqueous solution.

Preferably αB-crystallin or HSP17.5 are used for stabilizing luciferase. More preferably, αB-crystallin or HSP17.5 are used to stabilize luciferase at room temperature.

Use of HSP27, HSP25, αB-crystallin, α-crystallin, or αA-crystallin for stabilizing horseradish peroxidase (HRP) conjugate.

A method for stabilizing HRP conjugate in an aqueous solution comprising adding HSP27, HSP25, αB-crystallin, α-crystallin or αA-crystallin to said aqueous solution.

Preferably HSP27 and HSP25 are used to stabilize HRP conjugate at room temperature. Optionally, HSP27, HSP25 or αB-crystallin may be used to stabilize BRP conjugate at 37° C.

Use of HSP27 for stabilizing an antibody, antibody fragment or antibody conjugate.

A method for stabilizing conjugate thereof in an aqueous solution comprising adding HSP27 to said aqueous solution.

Preferably, HSP27 is used to stabilize an antibody at room temperature.

In a further aspect of the present invention there is provided a method of stabilizing an expressed recombinant protein comprising:
a) providing a cell capable of expressing said recombinant protein and a hybrid protein chaperone according to the present invention, and
b) expressing said recombinant protein and said hybrid protein chaperone in said cell.

In a yet further aspect of the present invention there is provided a cell capable of expressing a recombinant protein and a hybrid protein chaperone according to the present invention.

Examples of recombinant proteins according to the present invention may be understood to mean a recombinant protein and a hybrid protein chaperone according to the present invention.

In a further aspect of the present invention there is provided a nucleic acid sequence capable of encoding a hybrid protein chaperone according to the present invention.

In a further aspect of the present invention there is provided a vector comprising a nucleic acid sequence capable of encoding a hybrid protein chaperone according to the present invention. The vector may include vectors such as expression vectors known to the skilled man and as documented in Sambrook et al., 2001.

In yet a further aspect the vector of the present invention further comprises a nucleic acid sequence encoding a recombinant protein intended to be stabilized by the hybrid protein chaperone of the present invention as encoded by the nucleic acid sequence as hereinbefore described. The :recombinant protein is understood to mean the definition as hereinbefore described.

Embodiments of the invention will now be described, by way of example, with reference to the accompanying methods and figures, in which:

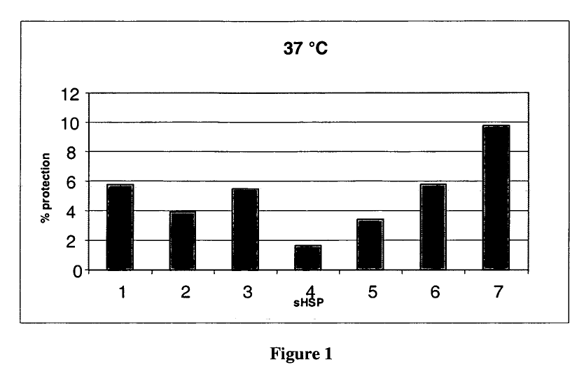

FIG. 1 shows a graph depicting results of an assay carried out at 37° C. on the protection of insulin against aggregation by protein chaperones, wherein 1. is α-crystallin, 2. is αA-crystallin, 3. is αB-crystallin, 4. is HSP20, 5. is HSP25, 6. is HSP27 and 7. is HSP17.5. Insulin with no addition of sHSP was taken as baseline and protection was calculated relative to this.

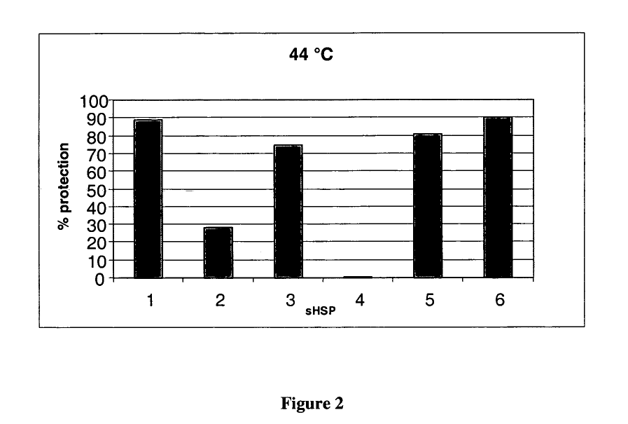

FIG. 2—as for FIG. 1 but depicting results of a similar insulin assay carried out at 44° C., wherein 1. is α-crystallin, 2. is αA-crystallin, 3. is αB-crystallin, 4. is HSP20, 5. is HSP25 and 6. is HSP27.

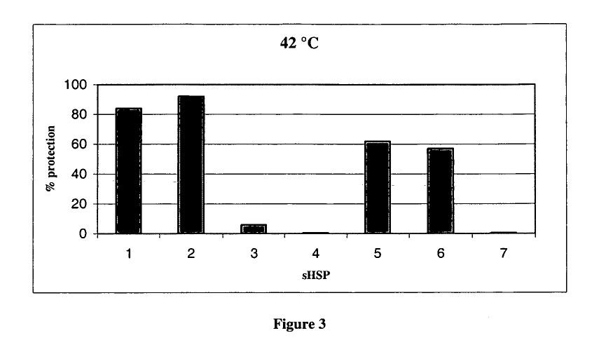

FIG. 3 shows a graph depicting the results of a citrate synthase assay performed at 42° C., wherein 1. is α-crystallin, 2. is αA-crystallin, 3. is αB-crystallin, 4. is HSP20, 5. is HSP25, 6. is HSP27 and 7. is No HSP. The results were calculated as for the insulin assay of FIGS. 1 and 2.

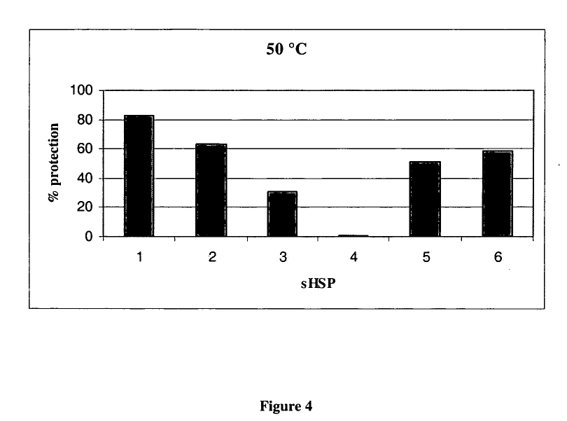

FIG. 4—as for FIG. 3 but depicting results of the citrate synthase assay carried out at 50° C., wherein 1. is α-crystallin, 2. is αA-crystallin, 3. is αB-crystallin, 4. is HSP20, 5. is HSP25, and 6. is HSP27.

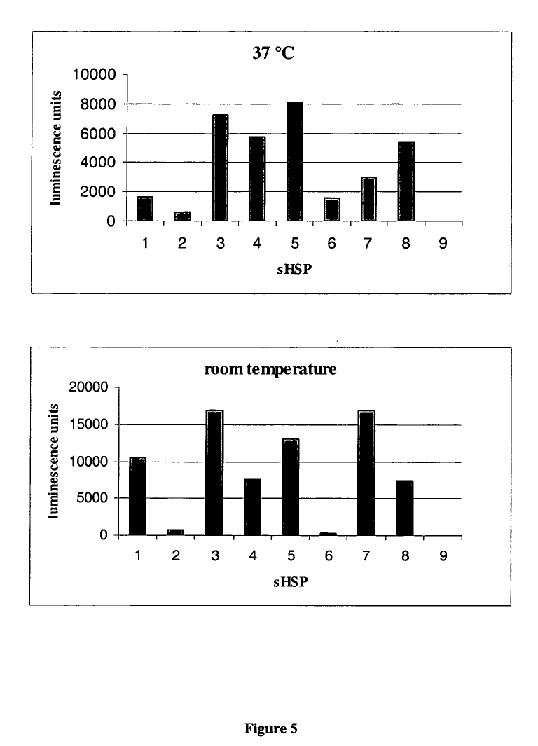

FIG. 5 shows a graph depicting results of an assay for luciferase after storage for one hour at 37° C. and 7 days at room temperature, wherein 1. is α-crystallin, 2. is αA-crystallin, 3. is αB-crystallin, 4. is HSP25, 5. is HSP27, 6. is HSP20, 7. is HSP17.5, 8. is BSA, and 9. is No HSP.

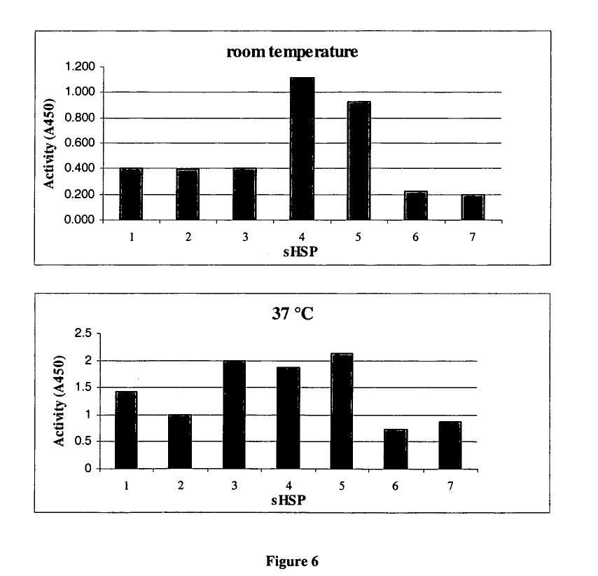

FIG. 6—shows a graph depicting results of an assay on HRP conjugate, after storage of the conjugates for 7 days at room temperature and one day at 37° C., wherein 1. is α-crystallin, 2. is αA-crystallin, 3. is αB-crystallin, 4. is HSP25, 5. is HSP27, 6. is BSA and 7. is No HSP.

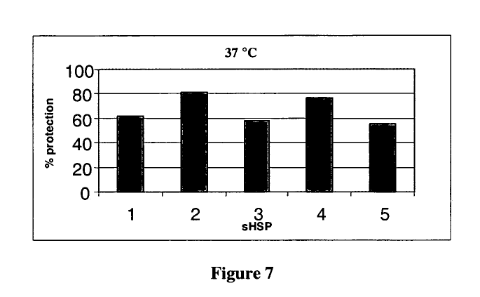

FIG. 7 shows a graph depicting the results of an assay carried out at 37° C. on the protection of insulin against aggregation by hybrid protein chaperone, wherein 1. is αB-crystallin, 2. is αB-MKBP, 3. is αB-αA, 4. is αB-HSP27, and 5. is αB-HSP17.5. Insulin with no addition of sHSP was taken as baseline and protection was calculated relative to this.

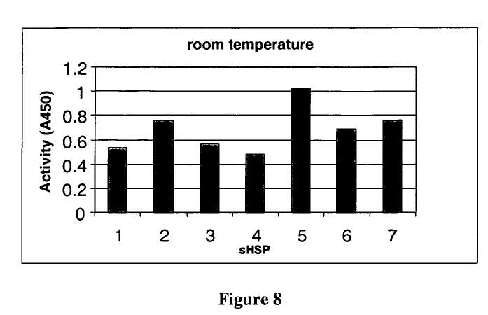

FIG. 8—as for FIG. 7 but depicting results of similar assay on HRP conjugate, after storage at room temperature for ten days, wherein 1. is no HSP, 2 is αB-crystallin, 3. is αB-MKBP, 4. is αB-αA, 5. is αB-HSP27, 6. is αB-HSP17.5, and 7 is HSP27.

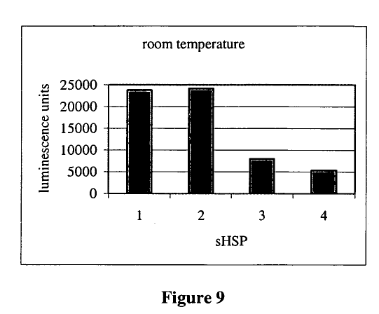

FIG. 9—as for FIG. 7 but depicting results of assay for luciferase, after storage at room temperature for 22 weeks, wherein 1. is αB-HSP27, 2. is αB-HSP17.5, 3. is αB-crystallin, and 4. is No HSP.

FIG. 10—shows protection of an antibody after three freeze thaws, wherein 1. is αB-αA, 2. is αB-HSP27, 3. is HSP27, and 4. is no ESP.

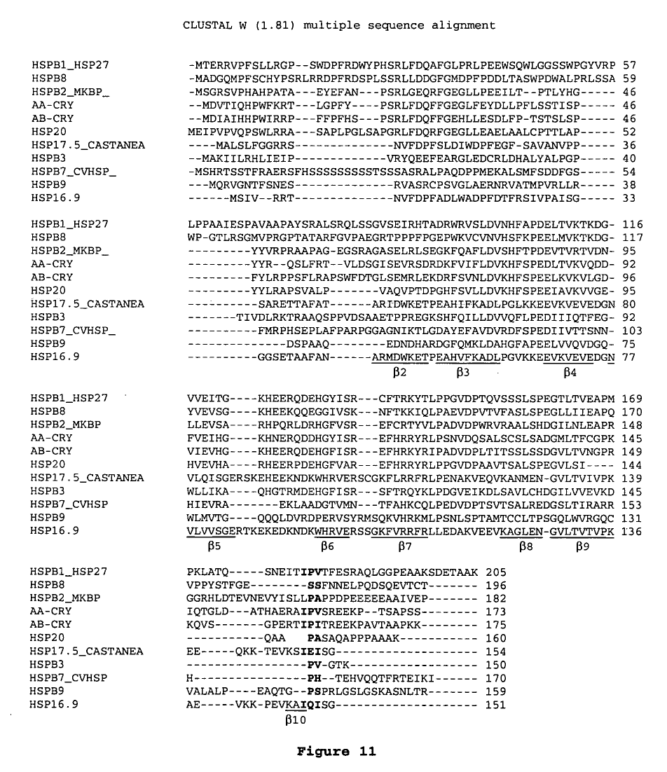

FIG. 11—shows a sequence alignment of eleven sHSPs (aligned further manually). β-strands for HSP16.9, displayed as lines below the alignment, as determined from the protein crystal structure (van Montfort et al. 2001). Accession numbers are HSPB1/HSP27 (P04792), HSPB8 (Q9UKS3), HSPB2/MKBP (Q16082), αA-crystallin (P02489), αB-crystallin (P02511), HSP20 (014558), HSP17.5 (AJ009880), HSPB3 (Q12988), HSPB7/CVHSP (Q9UBY9}, HSPB9 (AJ302068), and HSP16.9 (S21600).

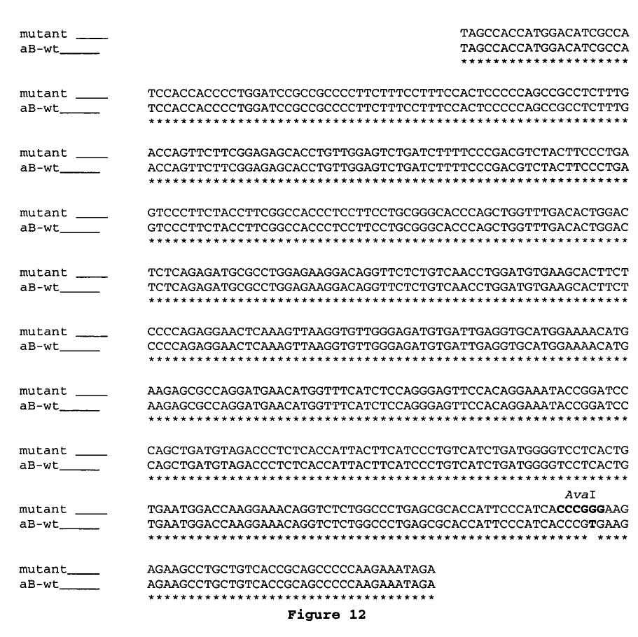

FIG. 12—depicts an alignment of sequences of αB-wildtype and mutant αB-crystallin. The mutation T489G is in bold. This is a silent mutation, which does not alter the protein sequence, but instead generates a unique AvaI site (underlined). This is used for subsequent cassette mutagenesis along with a vector based SacI site to introduce C-terminal sequences onto αB-crystallin.

### METHODS

A hybrid protein chaperone was constructed from αB-crystallin by swapping the αB-crystallin tail with the tail of a number of other sHSPs, including HSP27. sHSPS have short flexible and solvent exposed C-terminal extensions which protrude from the core of the molecule. The common characteristic of these extensions is that they are polar and are important for maintaining the solubility of the protein. Chimera were prepared as follows:

Cloning Strategy to Design Unique sHSPs by C-Tail Swapping.

The start of the tail was identified by sequence comparisons and multiple alignments of the sHSPs (FIG. 10). A general strategy was adopted to first introduce a unique restriction site at the C-tail junction followed by cassette mutagenesis to introduce new “tail” sequences.

Starting material: human αB-crystallin gene cloned into the bacterial expression vector pET23. The variable flexible C-tail of other human (HSP20, HSP27, MKBP, cvHSP and αA-crystallin) and plant (chestnut CsHSP17.5) sHSPs replaced the natural αρ-crystallin C-tail sequences by cassette mutagenesis. αB-crystallin wild type and αB-crystallin with no C-tail were used as controls.

The first step involved the introduction of a unique restriction enzyme site (AvaI) at the junction between the α-crystallin domain conserved among sHSPs and the variable C-tail domain by site directed mutagenesis of the human αB-Crystallin gene. A bacterial vector pGEMTeasy containing the αB-Crystallin coding sequence between the NcoI and EcoRI sites was used for the mutagenesis. We introduced the AvaI site at the site of a conserved amino acid (E164 in Accession No. P02511) close to the crystallin domain-c tail junction. This mutagenesis step led to the introduction of both a. silent coding mutation and the creation of an AvaI site at position 489 on the DNA sequence (FIG. 11). The expression vector contained a unique SacI site 3′ to the stop codon of αB-Crystallin. Oligonucleotides were designed to swap the αB-Crystallin C-tail sequence with that of other selected sHSPs. These were then inserted into the AvaI-SacI sites respectively of the αB-Crystallin expression construct. After production, the tails have been cloned into human αB-crystallin and DNA digested by AvaI-SacI. The different constructs were cloned into pET23d (Novagen) vector to perform the protein purification in E. coli BL21 plys strain. All the constructs work fine at the protein level.

Assay Principles

Insulin Assay

Insulin has 2 chains, A and B, linked by a disulphide band. Reduction, by DTT, destabilizes the protein conformation and induces aggregation. Aggregation is monitored by measurement of the absorbance at 360 nm for 10/15 minutes. The concentration of the insulin was 58 μm (equivalent to 350 μg/ml) and the ratio of insulin (Sigma I-5500) to sHSP was 4:1 (w:w). The assay was performed in a Beckman DU640 spectrophotometer at 37° C. and 44° C. The assay was based upon a published method as described (Farahbakhsh et al., 1995).

Citrate Synthase Assay

This follows the same principle as the insulin assay but is a thermal aggregation assay. The citrate synthase (Sigma #C-3260) was used at a concentration of 6 μm (equivalent to 300˜μg/ml) and the ratio of citrate synthase to sHSP was 4:1 (w:w using an assay as described in Buchner et al., 1998).

HRP Conjugate Assay

The assay measures the retention of enzyme activity and is therefore different to both the insulin and citrate synthase assays that are only a measure of protein stability.

This assay was developed using the innate instability of streptavidin-HRP (Sigma #S-9420) sourced from Sigma. Due to instability of this conjugate we have used it to test the efficacy of the sHSPs. The streptavidin-HRP conjugate loses activity when stored on the open bench and so a non-optimized assay was developed around this observation.

The assay uses a biotin coated plate (Pierce #15151) to capture the conjugate. Color development was by TMD substrate followed by addition of stop reagent and measurement of absorbance at 450 nm. The conjugate was stored at working strength (IU:6000 or 0.9 mg/ml) at room temperature and 37° C. and assayed at various time points to determine which sHSPs were chaperoning the protein most successfully. The sHSPs were added in a 40× weight excess.

Luciferase Assay

Luminescence is measured on addition of luciferin substrate and ATP substrate (Biothema Luciferase assay kit #-484-001) to the luciferase loaded in to a microtitre plate. The luminescence was measured by an Anthos Lucy 1 luminometer. Luciferase was stored at working strength at room temperature and 37° C. and activity monitored with time. This assay also measures the retention of enzyme activity.

### EXAMPLES

### Example 1

### Insulin Assay

The protection of the insulin against aggregation by the sHSPs is detailed in FIGS. 1 and 2. Insulin with no addition of sHSP was taken as baseline and protection was calculated relative to this.

This data shows that different sHSPs have different activities and that activity can be improved by mixing different chaperones, i.e. α-crystallin is better than αA-crystallin or αB-crystallin, αA-crystallin being worse than αB-crystallin. Also some sHSPs have no chaperone activity in this assay, e.g. HSP20. HSP27 and αB-crystallin both showed good chaperone activity and HSP17.5 performed the best. Prior art patent EP0599344A1 suggests the different sHSPs will have comparable activities, but these data do not support this assumption.

The data presented in FIG. 2 demonstrates that the sHSPs can show temperature variability, e.g. αB-crystallin. HSP25 and HSP27 all improve their relative activity at 44° C. compared to 37° C. These data refute previous claims made in the patent EP0599344A1. HSP27 and αB-crystallin again perform well with HSP27 being the better of the two.

### Example 2

### Citrate Synthase

The citrate synthase assay was performed at 42° C. and 50° C. and the results calculated as for the insulin assay. (FIGS. 3 and 4)

The citrate synthase results also show that sHSPs have different activities. In this assay, at both temperatures, αA-crystallin performed better than αB-crystallin. HSP20 was again inactive. HSP27's activity appeared constant at both 42° C. and 50° C., whereas αB-crystallin was less effective at 50° C. The naturally occurring mixture of αA/αB crystallin (α-crystallin) performed better than the individual proteins at 50° C.

### Example 3

### Luciferase

The luciferase activity was measured after storage for 1 hour at 37° C. and 7 days at room temperature (FIG. 5). From these studies it is clear that αB-crystalline and HSP17.5 are both proficient in preserving the enzyme activity of luciferase at room temperature. Once again there are differences between the individual chaperones with obvious poor (e.g. αA-crystallin, HSP20) as well as good chaperones. These data show how it is possible to extend the lifetime of the luciferase, which should then open up new applications for luciferase in the biodiagnostic industry. The inherent liability of the enzyme has restricted the application within applied biotechnology and consequently the market is currently limited to research applications. Chaperone addition can now open up new commercial applications of luciferase.

### Example 4

### HRP Conjugate

After incubation with substrate the absorbance at 450 nm was measured after storage of the conjugates for 7 days at room temperature and 1 day at 37° C. (FIG. 6). This is a measure of retained enzyme activity.

For the HRP conjugate HSP27 and HSP25 are the best for those stored at room temperature. At 37° C., αB-crystallin also shows similar activity to HSP25 and HSP27.

In a previous patent it was claimed that sHSPs, in general, would be effective at stabilizing proteins and protein activities. These data clearly demonstrate that individual sHSPs are better in some assays than in others. It is therefore difficult to select one natural sHSP chaperone to perform the best in all assays.

### Example 5

### Antibody Aggregation

An antibody known to aggregate and precipitate upon freeze thawing was used to assess the ability of the sHSPs to protect antibodies. 0.2 mg/ml of antibody was mixed with a ten fold weight excess of sHSP. The samples were then frozen in dry ice and thawed at room temperature for three freeze thaw cycles. Precipitated protein was removed by centrifugation at 10,000 g for 1 minute. The amount of non-aggregated antibody was determined by measuring the absorbance at 280 nm. Results are shown in FIG. 10 and discussed below in Example 6.

### Example 6

A hybrid protein chaperone was constructed from αB-crystallin by swapping the αB-crystallin tail with the tail of a number of other sHSPs, including HSP27. The hybrids were tested with insulin (FIG. 7), the HRP conjugate, after storage at room temperature for ten days (FIG. 8) and luciferase, after storage at room temperature for 22 weeks (FIG. 9). The ability of sHSP hybrids to protect antibodies against repeated freeze thaw induced precipitation was also tested (FIG. 10).

In the insulin assay αB-crystallin with the MKBP and HSP27 tails both show enhanced activity compared to αB-crystallin. Similarly for the HRP conjugate and luciferase αB-HSP27 chaperones the HRP conjugate more effectively than either αB-crystallin or HSP27. For the antibody protection a similar result was obtained with HSP27 and αB-crystallin hybrids. The hybrids had greater activity compared to HSP27. These data show that the hybrid sHSP, αB-HSP27 can protect all four substrates tested here and was the best in each case.

### REFERENCES

Andley, U. P., Z. Song, E. F. Wawrousek, J. P. Brady, S. Bassnett, and T. P. Fleming, 2001. Lens epithelial cells derived from alphaB-crystallin knockout mice demonstrate hyperproliferation and genomic instability. Faseb J. 15: 221-229.
Aurelian, L., C. C. Smith, R. Winchurch, M. Kulka, T. Gyotoku, L. Zaccaro, F. J. Chrest, and J. W. Burnett. 2001. A novel gene expressed in human keratinocytes with long-term in vitro growth potential is required for cell growth. J. Invest. Dermatol. 226: 286-95.
Berry, V., P. Francis, M. A. Reddy, D. Collyer, E. Vithana, R. MacKay, G. Dawson, A. H. Carey, A. Moore, S. S. Bhattacharya, and R. A. Quinlan. 2001. Alpha-b crystallin gene (cryab) mutation causes dominant congenital posterior polar cataract in humans. Am J Hum Genet, 69: 1141-5.
Buchner, J., H. Grallert, and U. Jakob. 1998. Analysis of chaperone function using citrate synthase as normative substrate protein. Methods Enzymol. 290: 323-38.
de Jong, W. W., G. J. Caspers, and J. A. Leunissen. 1998. Genealogy of the alpha-crystallin—small heat-shock protein superfamily. Int J Biol Macromol. 22: 151-62.
Ellis, R. J., and F. U. Hartl. 1999. Principles of protein folding in the cellular environment [In Process Citation], Curr Opin Struct Biol. 9; 102-10.
Farahbakhsh, Z. T., Q. L. Huang, L. L. Ding, C. Altenbach, H. J. Steinhoff, J. Horwitz, and W. L. Hubbell, 1995. Interaction of alpha-crystallin with spin-labeled pepcides. BioChemistry 34: 509-16.
Hatters, D. M., R. A. Lindner, J. A. Carver, and G. J. Howlett. 2001. The molecular chaperone, alpha-crystallin, inhibits amyloid formation by apolipoprotein C-II. J. Biol. Chem. 276; 33755-61.
Kamradt, M. C., F. Chen, and V. L. Cryns, 2001. The small heat shock protein alpha B-crystallin negatively regulates cytochrome c- and caspase-8-dependent activation of caspase-3 by inhibiting its autoproteolytic maturation. J Biol. Chem. 276: 16059-63.
Kappe, G., :P. Verschuure, P. Van de Boogaart, R. L. Philipsen, A. A. Staalduinen, W. C. Boelens, and W. W. De Jong. 2001. Characterization of two novel human small heat shock proteins: protein kinase-related HspB8 and testis-specific HspB9. Biochim Biophys Acta. 1520: 2-6.
Kim, K. K.; R. Kim, and S. H. Kim. 1998. Crystal structure of a small heat-shock protein. Nature. 394: 595-599.
Krief; S., J. F. Faivre, P. Robert, B. Le Douarin, N. Brument-Larignon, I. Lefrere, M. M. Bouzyk, K. M. Anderson, L. D. Greller, P. L. Tobin, M. Souchet, and A. Bril. 1999. Identification and characterization of cvHsp. A novel human small stress protein selectively expressed in cardiovascular and insulin-sensitive tissues. J Biol. Chem. 274: 36592-600.
Kudva, Y. C., H. J. Hiddinga, P. C. Butler, C. S. Mueske, and N. L. Eberhardt. 1997. Small heat shock proteins inhibit in vitro A beta(1-42) amyloidogenesis. FEBS LecE. 416: 117-21.
Lindner, R. A., J. A- Carver, M. Ehrnsperger, J. Buchne˜, G. Esposito, .J. Behlke, G. Lutsch, A. Kotlyarov, and M. Gaestel. 2000. Mouse Hsp25, a small shock protein. The role of its c-terminal extension in oligomerization and chaperone action—Bur J. Biochem. 267; 2923-32.
Lindner, R. A. T. M. Treweek, and J. A. Carver, 2001. The molecular competition chaperone alpha-crystallin is in kinetic compeitition with aggregation to stabilize a monomeric molten-globule form of alpha-lactalbumin. Biochem J. 354: 79-87.
Litt, M., P. Kramer, D. M. Lamorticella, W. Murphey, E. W. Lovrien, and R. G. Weleber, 1998. Autosomal dominant congenital cataract associated with a missense mutation in the human alpha crystallin gene CRYAA. Hum Mol Genet. 7: 471-4.
Mehlen, P., K. Schulzeosthoff, and A. P. Arrigo. 1996. Small stress proteins as novel regulators of apoptosis-heat-shock protein-27 blocks fas/apo-1-induced and staurosporine-induced cell-death. J Biol Chem, 271: 16510-16514.
Niwa, M., O. Kozawa, E- Matsuno, K. Kato, and T. Uematsu. 2000. Small molecular weight heat shock-related protein, HSP20, exhibits an anti-platelet activity by inhibiting receptor-mediated calcium influx. Life Sci. 66: L7-12.
Perng, M. D., L. Cairns, P. van den IJssel, A. Prescott, A. M. Hutcheson, and R. A. Quinlan. 1999a. Intermediate filament interactions can be altered by HSP27 and αB-crystallin. J Cell. Sci. 112: 2099-2112.
Perng, M. D., .J. Muchowski, P. van den IJssel, G. J. S. Wu, J. I. Clark, and R. A. Quinlan. 1999b. The cardiomyopathy and lens cataract mutation in αB-crystallin compromises secondary, tertiary and quaternary protein structure and reduces in vitro chaperone activity. J Biol. Chem. 274: 33235-43.
Pietrowski, D., M. J. Durante, A. Liebstein, T. Schmitt-John, T. Werner, and J. Graw. 1994. Alpha-crystallins are involved in specific interactions with the murine gamma D/E/F-crystallin-encoding gene. Gene. 144: 171-8.
Reddy, G. B., K. P. Das, J. M. Petrash, and W. K. Surewicz. 2000. Temperature-dependent chaperone activity and structural properties of human alphaA- and alphaB-crystallins. J Biol Chem. 275: 4565-70.
Saibil, H. 2000. Molecular chaperones: containers and surfaces for folding; stabilizing or unfolding proteins. Curr. Opin. Struct. Biol., 10: 251-8.
Sambrook, J., Russell, D. W., and Sambrook, J., 2001. Molecular Cloning: A laboratory Manual. Cold Spring Harbor Labor˜tory Press; 3rd edition.
Schubert, U., L. C. Anton, J. Gibba, C. C. Norbury, J. W. Yewdell, and .T. R. Bennink. 2000. Rapid degradation of a large fraction of newly synthesized proteins by proteasomes. Nature, 404: 770-4.
Suzuki, A., Y. Sugiyama, Y. Hayashi, N. Nyu-i, M. Yoshida, I. Nonaka, S. Ishiura, K. Arahata, and S. Ohno. 1998. MKBP, a novel member of the small heat shock protein family, binds and activates the myotonic dystrophy protein kinase. J Cell Biol. 140: 1113-24.
van de Klundert, F. A., R. H. Smulders, M. L. Gijsen, R. A. Lindner, R. Jaenicke, J. A. Carver, and W. W. de Jong, 1998. The mammalian small heat-shock protein Hsp20 forms dimers and is a poor chaperone. Eur J Biochem. 258: 1014-21.
van Montfort, R. L., E. Basha, K. L. Friedrich, C. Slingsby, and E. Vierling. 2001. Crystal structure and assembly of a eukaryotic small heat shock protein. Nat Struct Biol. 8: 1025-30.
Vicart, P., A. Caron, P. Guicheney, Z. Li, M. C. Prevost, A. Faure, D. Chateau, F. Chapon, F. Tome, J. M. Dupret, D. Paulin, and M. Fardeau, 1998. A missense mutation in the alphaB-crystallin chaperone gene causes a desmin-related myopathy. Nat Genet. 20; 92-5.
Kappe, G., Leunissen and W. W. de Jong, 2002. Evolution and Diversity of Prokaryotic Small Heat Shock Proteins. Progress in Molecular and Subcellular Biology 28: 1-17.

1. A hybrid protein chaperone for stabilizing proteins and/or protein activities.

2. The hybrid protein chaperone according to claim 1, wherein the hybrid is a macromolecule composed of two or more portions of different origins.

3. The hybrid protein chaperone according to claim 2, wherein the portion is a region of protein or nucleic acid sequence encoding a structural domain of a protein chaperone or functional homologue thereof.

4. The hybrid protein chaperone according to claim 3, wherein the structural domain is a central domain, N- or C-terminal region of a protein chaperone or functional homologue thereof.

5. The hybrid protein chaperone according to claim 4, wherein the protein chaperone is a heat shock protein.

6. The hybrid protein chaperone according to claim 5, wherein the heat shock protein is selected from the group consisting of HSP90, HSP70 and HSP60.

7. The hybrid protein chaperone according claim 1, wherein the protein chaperone is a small heat shock protein (sHSP).

8. The hybrid protein chaperone according to claim 7, wherein the sHSP is selected from the group consisting of αA-crystallin, αB-crystallin, HSP27, HSP20, MKBP, HSPB3, HSPB4, HSPB5, cvHSP, HSPB8 and HSPB9.

9. The hybrid protein chaperone according to claim 8, wherein the portions comprise sub-domains or residues of sHSP.

10. The hybrid protein chaperone according to claim 9, wherein the residue is Arginine 120.

11. The hybrid protein chaperone according to claim 9, wherein the sub-domain is the C-terminal region.

12. The hybrid protein chaperone according to claim 1, wherein a portion of the chaperone is replaced with a similar portion from a chaperone of a different origin.

13. The hybrid protein chaperone according to claim 8, wherein a C-terminal portion of αB-crystallin is replaced with a C-terminal portion of HSP27 (αB-HSP27).

14. The hybrid protein chaperone according to claim 13, wherein αB-HSP27 comprises the N-terminus and central portion of αB-crystallin and C-terminal tail of HSP27.

15. A stabilized protein formulation comprising at least one protein associated with a hybrid protein chaperone according to claim 1.

16. The stabilized protein formulation according to claim 15, wherein the ratio of protein to hybrid protein chaperone in the formulation is in the region of 25:1 to 1:100.

17. The stabilized protein formulation according to claim 15, wherein the ratio of protein to hybrid protein chaperone in the formulation is 1:0.0625 to 1:40.

18. A method for stabilizing proteins and protein stabilities in an aqueous solution comprising adding the hybrid protein chaperone according to claim 1 to the aqueous solution.

19. The method according to claim 18, wherein the protein to be stabilized is an enzyme, therapeutic protein, diagnostic protein, antibody, antibody fragment or antibody conjugate.

20. The method according to claim 19, wherein the protein is homocysteine desulphurase.

21. The method according to claim 19, wherein the antibody conjugate is covalently linked to an enzyme reporter.

22. The method according to claim 21, wherein the enzyme reporter is horseradish peroxidase (HRP), alkaline phosphatase (ALP), or luciferase.

23. The method according to claim 18, wherein stabilizing is the prevention or arresting of the unfolding process and preservation of protein activity/function.

24. The method according to claim 23, wherein the preservation of protein activity/function is achieved by assisting proteins to fold correctly and maintaining the proteins in a folded conformation.

25. The method according to claim 18, wherein the hybrid protein chaperone is αB-crystallin and the protein is luciferase.

26. The method according to claim 18, wherein the hybrid protein chaperone is αB-HSP27 and the protein is insulin, HRP conjugate or luciferase.

27. The method according to claim 18, wherein the hybrid protein chaperone prevents protein aggregation.

28. The method according to claim 23, wherein the prevention or arresting further inhibits cell death.

29. A method for stabilizing insulin in an aqueous solution comprising adding HSP17.5, α-crystallin, HSP27, αB-crystallin, αA-crystallin or HSP25 to the solution.

30. The method according to claim 29, wherein HSP17.5 is used to stabilize insulin at 37° C.

31. The method according to claim 29, wherein HSP27 or α-crystallin is used to stabilize insulin at 44° C.

32. A method for stabilizing citrate synthase in an aqueous solution comprising adding αA-crystallin, αB-crystallin, α-crystallin, HSP25 or HSP27 to the aqueous solution.

33. The method according to claim 32, wherein α-crystallin is used to stabilize citrate synthase at 50° C.

34. A method for stabilizing luciferase in an aqueous solution comprising adding HSP17.5, HSP27 or α-crystallin to the aqueous solution.

35. The method according to claim 34, wherein αB-crystallin or HSP17.5 are added to stabilize luciferase at room temperature.

36. A method for stabilizing HRP conjugate in an aqueous solution comprising adding HSP27, HSP25, αB-crystallin, α-crystallin or αA-crystallin to the aqueous solution.

37. The method according to claim 36, wherein HSP27 and HSP25 are added to the aqueous solution to stabilize HRP conjugate at room temperature.

38. The method according to claim 36, wherein HSP27, HSP25 or αB-crystallin are added to stabilize HRP conjugate at 37° C.

39. A method for stabilizing an antibody, fragment or conjugate thereof in an aqueous comprising adding HSP27 to the aqueous solution.

40. The method according to claim 39, wherein HSP27 is used to stabilize an antibody at room temperature.

41. A method of stabilizing an expressed recombinant protein comprising:
a) providing a cell capable of expressing the recombinant protein and a hybrid protein chaperone according to claim 1; and
b) expressing the recombinant protein and the hybrid protein chaperone in the cell.

42. The method according to claim 41, wherein the recombinant protein is a therapeutically important protein.

43. A cell capable of expressing a recombinant protein and a hybrid protein chaperone according claim 1.

44. A nucleic acid sequence capable of encoding a hybrid protein chaperone according claim 1.

45. A vector comprising the nucleic acid sequence of claim 44.

46. The vector according to claim 45, further comprising a nucleic acid capable of encoding a recombinant protein intended to be stabilized by the hybrid protein chaperone.
